(19) United States
(12) Patent Application Publication
(10) Pub. No.: US 2021/0186838 A1
(43) Pub. Date: Jun. 24, 2021

(54) ACTIVE SUBSTANCE COMBINATIONS OF N-(4-AMINO-2-METHYLQUINOLIN-6-YL)-2-((4-ETHYLPHENOXY)METHYL)BENZAMIDE AND ONE OR MORE COSMETICALLY OR DERMATOLOGICALLY ACCEPTABLE PRESERVATIVES AND/OR PRESERVATIVE AUXILIARIES

(71) Applicant: BEIERSDORF AG, Hamburg (DE)

(72) Inventors: Wilfried SIEFKEN, Hamburg (DE); Joern Hendrik REUTER, Henstedt Ulzburg (DE); Kerstin SKUBSCH, Prisdorf (DE)

(21) Appl. No.: 16/633,178

(22) PCT Filed: May 25, 2018
(86) PCT No.: PCT/EP2018/063797

§ 371 Date: Jan. 23, 2020

(30) Foreign Application Priority Data
Jul. 24, 2017 (DE) 10 2017 212 647.7

(51) Int. Cl. A61K 8/49; A61Q 19/08; A61K 8/06; A61Q 17/04

(52) U.S. Cl. CPC A61K 8/4926; A61Q 19/08; A61K 2800/524; A61Q 17/04; A61K 8/06

(57) ABSTRACT

Active substance combinations of N-(4-amino-2-methylquinoline-6-yl)-2-((4-ethylphenoxy)methyl)benzamide and one or more cosmetically or dermatologically acceptable preservatives and/or preservative auxiliaries.

The present invention relates to cosmetic and dermatological preparations containing N-(4-amino-2-methylquinolin-6-yl)-2-((4-ethylphenoxy)methyl)benzamide for the care and protection of the skin, in particular sensitive skin and, above all, skin aged by intrinsic and/or extrinsic factors or aging skin, and also to the use of such active substances and combinations of such active substances in the field of cosmetic and dermatological skincare.

Cosmetic skincare is to be understood as meaning, first and foremost, strengthening or restoring the skin's natural function as a barrier against environmental influences (for example dirt, chemicals, microorganisms) and against the loss of endogenous substances (for example water, natural fats, electrolytes).

Impairment of this function can lead to increased absorption of toxic or allergenic substances or colonization by microorganisms, resulting in toxic or allergic skin reactions.

With old skin, for example, regenerative renewal is slower, this being reflected in particular in a decrease in the water-binding capacity of the stratum corneum. It consequently becomes inflexible, dry, and cracked (“physiologically” dry skin). Barrier damage is the result. The skin becomes susceptible to adverse environmental influences such as invasion by microorganisms, toxins, and allergens. This can result even in toxic or allergic skin reactions.

In the case of pathologically dry and sensitive skin, barrier damage is a given. The formation of epidermal intercellular lipids is defective or inadequate in quantity or composition. The consequence is increased permeability of the stratum corneum and insufficient protection of the skin from loss of hygroscopic substances and water.

The barrier action of the skin can be quantified by determining the transepidermal water loss (TEWL). This is the evaporation of water from inside the body, not including water loss while sweating. Quantitation of the TEWL has been found to be extremely informative and can be used in the diagnosis of cracked or chapped skin, to determine the tolerability of surfactants with different chemical structures, and so on.

Chronological aging of the skin is caused for example by endogenous, genetically determined factors. In the epidermis and dermis, age-related processes result, for example, in the following structural damage and functional disturbances, which can also be covered by the term “senile xerosis”:

a) dryness, roughness, and development of fine wrinkles caused by dryness,
b) itching, and
c) decreased refatting by sebaceous glands (for example after washing).

Exogenous factors, such as UV light and chemical toxins, can have a cumulative effect and, for example, accelerate the endogenous aging process or add to it. In the epidermis and dermis, exogenous factors in particular result, for example, in the following structural damage and functional impairment in the skin, which outstrip the damage from chronological aging in both extent and nature:

d) visible vasodilation phenomena (telangiectasias, couperose);
e) sagging skin and development of wrinkles;
f) local hyper-, hypo-, and dyspigmentation (for example age spots), and
g) increased susceptibility to mechanical stress (for example cracking).

The present invention relates in particular to products for the care of naturally aged skin and for treating the secondary damage from photoaging, in particular the phenomena mentioned under a) to g).

Products for the care of aged skin are known per se. They contain, for example, retinoids (vitamin A acid and/or derivatives thereof) or vitamin A and/or derivatives thereof. However, their effect on structural damage is of limited extent. What is more, during product development there are considerable difficulties in stabilizing the active substances sufficiently against oxidative degradation. The use of products containing vitamin A acid often, moreover, causes severe erythematous skin irritation. Retinoids can therefore be used only in low concentrations.

In particular, the present invention relates to cosmetic preparations providing effective protection against harmful oxidation processes in the skin, but also to the protection of cosmetic preparations themselves, and to the protection of constituents of cosmetic preparations, against harmful oxidation processes.

The present invention further relates to antioxidants, preferably those that are used in cosmetic or dermatological skincare preparations. In particular, the invention relates also to cosmetic and dermatological preparations containing such antioxidants. In a preferred embodiment, the present invention relates to cosmetic and dermatological preparations for the prophylaxis and treatment of cosmetic or dermatological skin changes such as skin aging, in particular skin aging caused by oxidative processes.

In addition, the present invention relates to active substances and preparations containing such active substances for the cosmetic and dermatological treatment or prophylaxis of erythematous, inflammatory, allergic or autoimmune reactive phenomena, especially dermatoses.

In a further advantageous embodiment, the present invention relates to combinations of active substances and to preparations used for the prophylaxis and treatment of photosensitive skin, especially photodermatoses.

The damaging effect on the skin of the ultraviolet component of sunlight is generally known. Whereas rays having a wavelength of less than 290 nm (the so-called UVC range) are absorbed by the ozone layer in the earth's atmosphere, rays in the range between 290 nm and 320 nm, the so-called UVB range, cause erythema, simple sunburn or actual burns of varying severity.

The erythematous activity of sunlight is said to peak in the narrower range around 308 nm.

Numerous compounds are known to protect against UVB radiation, these being derivatives of 3-benzylidenecamphor, 4-aminobenzoic acid, cinnamic acid, salicylic acid, benzophenone, and also 2-phenylbenzimidazole.

For the range between about 320 nm and about 400 nm, the so-called UVA range, it is likewise important for there to be filter substances available, since such radiation can cause reactions in photosensitive skin. UVA radiation has been shown to result in damage to elastic and collagenous fibers in connective tissue, which causes the skin to age prematurely and can also be regarded as the cause of numerous phototoxic and photoallergic reactions. The damaging effect of UVB radiation can be intensified by UVA radiation.

To protect against radiation in the UVA range, certain derivatives of dibenzoylmethane are accordingly used, the photostability of which is insufficiently established (Int. J. Cosm. Science 10, 53 (1988)).

UV radiation can however also result in photochemical reactions; if this happens, the products of these photochemical reactions interfere in the skin metabolism.

Such photochemical reaction products are predominantly free-radical compounds, for example hydroxyl radicals, singlet oxygen. Undefined free-radical photodegradation products that are formed in the skin itself can show uncontrolled secondary reactions due to the high reactivity thereof. However, singlet oxygen, a non-free radical, excited state of the oxygen molecule, can also arise during UV irradiation, as can short-lived epoxides and many other species. Singlet oxygen, for example, is characterized by increased reactivity compared with the triplet oxygen (free-radical ground state) that is normally present. However, excited, reactive (free-radical) triplet states of the oxygen molecule also exist.

UV radiation also ranks as ionizing radiation. During exposure to UV, there is therefore also a risk of formation of ionic species, which could in turn then act as oxidants in biochemical processes.

To prevent such reactions, additional antioxidants and/or free-radical scavengers can be incorporated into cosmetic or dermatological formulations.

The use of vitamin E, a substance with a known antioxidant effect in light-protection formulations, has previously been proposed, but the achieved effect here too falls well short of what had been hoped for.

The object of the invention was therefore also to provide cosmetic, dermatological, and pharmaceutical active substances and preparations and to create light-protection formulations that are used for the prophylaxis and treatment of light-sensitive skin, in particular photodermatoses, primarily PLE.

Other expressions for polymorphous light eruption are PLE, PLME, Mallorca acne, and a variety of other expressions used in the literature (for example A. Voelckel et al, Zentralblatt Haut- and Geschlechtskrankheiten (1989), 156, p. 2).

Antioxidants are used mainly as substances to protect preparations in which they are present from deterioration. Undesirable oxidation processes are nevertheless known to occur in human and animal skin too. Such processes play a key role in skin aging.

The article “Skin Diseases Associated with Oxidative Injury” in “Oxidative Stress in Dermatology”, pp. 323 ff. (Marcel Decker Inc., New York, Basel, Hong Kong, editors: Jürgen Fuchs, Frankfurt, and Lester Packer, Berkeley/California) details oxidative damage to the skin and the more immediate causes thereof.

For the purposes also of preventing such reactions, antioxidants and/or free-radical scavengers can be additionally incorporated into cosmetic or dermatological formulations.

Some antioxidants and free-radical scavengers are certainly known. The use of vitamin E, a substance with a known antioxidant effect in light-protection formulations, has previously been proposed in U.S. Pat. Nos. 4,144,325 and 4,248,861 and in numerous other documents, but the achieved effect here too falls well short of what had been hoped for.

The object of the present invention was therefore to find ways of avoiding the disadvantages of the prior art. In particular, the effect of repairing the damage associated with endogenous, chronological, and exogenous skin aging and the prophylaxis thereof needs to be long-lasting, sustained, and without the risk of side effects.

According to the invention, the shortcomings of the prior art are eliminated by active substance combinations of N-(4-amino-2-methylquinolin-6-yl)-2-((4-ethylphenoxy)methyl) benzamide and one or more cosmetically or dermatologically acceptable preservatives or preservative auxiliaries.

Cosmetic or dermatological preparations according to the invention, active substance combinations of N-(4-amino-2-methylquinolin-6-yl)-2-((4-ethylphenoxy)methyl) benzamide and one or more cosmetically or dermatologically acceptable preservatives or preservative auxiliaries, are in every respect extremely satisfactory preparations. It was not possible for those skilled in the art to predict whether the preparations according to the invention would be
better at maintaining or restoring the skin's barrier properties,
better at counteracting dehydration,
more effective against pigmentation disorders,
more effective against skin aging, and
better at protecting the skin from environmental influences
   than the preparations of the prior art.

Surprisingly, when using active substance and cosmetic or topical dermatological preparations employed according to the invention that have an effective content of active substance combinations employed according to the invention, not only is effective treatment, but also prophylaxis
of deficient, sensitive or hypoactive skin states or deficient, sensitive or hypoactive states of skin appendages
of signs of premature aging of the skin (for example wrinkles, age spots, telangiectasias) and/or of skin appendages,
of environmental (smoking, smog, reactive oxygen species, free radicals) and in particular light-related adverse changes to the skin and skin appendages.
of light-related skin damage
of pigmentation disorders,
of itching,
of dry skin states and disorders of the stratum corneum barrier,
of hair loss and for boosting hair growth
of inflammatory skin conditions and also atopic dermatitis, seborrheic dermatitis, polymorphous light eruption, psoriasis, vitiligo
   possible. The active substance and cosmetic or topical dermatological preparations according to the invention that have an effective content of active substance according to the invention may surprisingly also be used
to soothe sensitive or irritated skin
to stimulate the synthesis of collagen, hyaluronic acid, and elastin
to stimulate ceramide synthesis in the skin
to stimulate intracellular DNA synthesis, particularly in deficient or hypoactive skin states.
to boost cell renewal and regeneration in the skin
to boost the skin's own protection and repair mechanisms (for example for dysfunctional enzymes, DNA, lipids, proteins)
for treatment before and after topical application of laser and abrasive treatments that are used for example for reducing skin wrinkles and scars, to counteract the resulting skin irritation and to promote regeneration processes in the injured skin.

N-(4-Amino-2-methylquinolin-6-yl)-2-((4-ethylphenoxy)methyl)benzamide is characterized by the structural formula

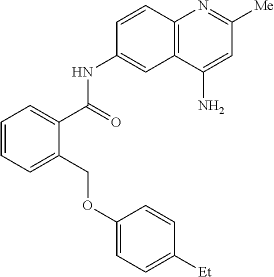 [SMILES: CCc1ccc(OCc2ccccc2C(=O)Nc2ccc3nc(C)cc(N)c3c2)cc1]

Cosmetic or dermatological preparations according to the invention preferably contain 0.001-10% by weight, more preferably 0.01-1% by weight, of N-(4-amino-2-methylquinolin-6-yl)-2-((4-ethylphenoxy)methyl)benzamide based on the total composition of the preparations.

In order to provide cosmetic preparations, cosmetic or dermatological preparations as used according to the invention advantageously comprise one or more preservatives or preservative auxiliaries, wherein the total amount of preservatives or preservative auxiliaries is, for example, 0.01% by weight to 20% by weight, preferably 0.1% to 20% by weight, more preferably 0.5% to 5.0% by weight, based on the total weight of the preparations.

The preparations according to the invention advantageously contain 0.01-20% by weight of one or more preservatives or preservative auxiliaries, preferably 0.1-5% by weight of one or more preservatives or preservative auxiliaries, in particular methyl 4-hydroxybenzoate, ethylparaben, phenoxyethanol, piroctone olamine, methylisothiazolinone, methylpropanediol, butylene glycol, caprylyl glycol, octane-1,2-diol, ethylhexylglycerol, glyceryl caprylate, propylene glycol, sodium salicylate, sodium benzoate, pentylene glycol, 1,2-hexanediol, DMDM hydantoin, benzyl alcohol, butylene glycol, iodopropynyl butylcarbamate.

It is additionally advantageous according to the invention if the preparation contains propylene glycol, butylene glycol, 2-methylpropane-1,3-diol, ethylhexylglycerol, 1,2-pentanediol, 1,2-hexanediol, 1,2-octanediol, and/or 1,2-decanediol.

The use of ethylhexylglycerol is according to the invention preferable.

According to the invention, the use of ethylhexylglycerol at a concentration of 0.1 to 0.6% by weight based on the total weight of the preparation is advantageous.

The content of phenoxyethanol in the preparation is according to the invention likewise advantageous.

According to the invention, the use of phenoxyethanol at a concentration of 0.1 to 1% by weight based on the total weight of the preparation is advantageous.

The use of a combination of phenoxyethanol and ethylhexylglycerol is according to the invention particularly preferred.

In order to provide cosmetic preparations that protect the hair and skin from the entire range of ultraviolet radiation, the preparations according to the invention advantageously comprise substances that absorb UV radiation in the UV-A and/or UV-B range, wherein the total amount of filter substances is, for example, 0.1% by weight to 40% by weight, preferably 1 to 30% by weight, more preferably 3 to 20% by weight, based on the total weight of the preparations. They can also serve as sunscreens for the hair or skin.

The UV-B filters may be oil-soluble or water-soluble. Examples of advantageous oil-soluble UV-B filter substances are:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
2,4,6-tri[anilino-(p-carbo-2′-ethyl-1′-hexyloxy)]-1,3,5-triazine;
esters of benzalmalonic acid, preferably di(2-ethylhexyl) 4-methoxybenzalmalonate;
esters of cinnamic acid, preferably 2-ethylhexyl 4-methoxycinnamate, isopentyl 4-methoxycinnamate;
derivatives of benzophenone, preferably 2-hydroxy-4-m ethoxybenzophenone, 2-hydroxy-4-methoxy-4′-methylbenzophenone, 2,2′-dihydroxy-4-methoxybenzophenone
and also UV filters bound to polymers.

Examples of advantageous water-soluble UV-B filter substances are:
salts of 2-phenylbenzimidazole-5-sulfonic acid, such as the sodium, potassium or triethanolammonium salt thereof and also the sulfonic acid itself;
sulfonic acid derivatives of 3-benzylidenecamphor, such as 4-(2-oxo-3-bornylidenemethyl)benzenesulfonic acid, 2-methyl-5-(2-oxo-3-bornylidenemethyl)sulfonic acid, and salts thereof.

The list of cited UV filters that may be used in the context of the present invention is of course not intended to be limiting.

It may be advantageous according to the invention to use customary antioxidants in preparations comprising active substance combinations according to the invention.

The antioxidants are advantageously selected from the group consisting of amino acids (for example glycine, histidine, tyrosine, tryptophan) and derivatives thereof, imidazoles (for example urocanic acid) and derivatives thereof, peptides such as DL-carnosine, D-carnosine, L-carnosine and derivatives thereof (for example anserine), carotenoids, carotenes (for example α-carotene, β-carotene, lycopene) and derivatives thereof, aurothioglucose, propylthiouracil and other thiols (for example thioredoxin, glutathione, cysteine, cystine, cystamine, and the glycosyl, N-acetyl, methyl, ethyl, propyl, amyl, butyl and lauryl, palmitoyl, oleyl, γ-linoleyl, cholesteryl, and glyceryl esters thereof) and salts thereof, dilauryl thiodipropionate, distearyl thiodipropionate, thiodipropionic acid and derivatives thereof (esters, ethers, peptides, lipids, nucleotides, nucleosides, and salts) and also sulfoximine compounds (for example buthionine sulfoximine, homocysteine sulfoximine, buthionine sulfones, pentathionine sulfoximine, hexathionine sulfoximine, heptathionine sulfoximine) in very low tolerated doses (for example pmol to μmol per kg), also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, alanine diacetic acid, flavonoids, polyphenols, catechins, vitamin C and derivatives (for example ascorbyl palmitate, Mg ascorbyl phosphate, ascorbyl acetate), tocopherols and derivatives (for example vitamin E acetate), and also coniferyl benzoate of benzoin resin, rutic acid and derivatives thereof, ferulic acid and derivatives thereof, butylated hydroxytoluene, butylated hydroxyanisole, nordihydroguaiaretic acid, trihydroxy-butyrophenone, uric acid and derivatives thereof, mannose and derivatives thereof, zinc and derivatives thereof (for example ZnO, ZnSO₄), selenium and derivatives thereof (for example selenium methionine), stilbenes and derivatives thereof (for example stilbene oxide, trans-stilbene oxide), and appropriate derivatives according to the invention (salts, esters, ethers, sugars, nucleotides, nucleosides, peptides, and lipids) of these recited active substances.

The amount of antioxidants (one or more compounds) in the preparations is preferably 0.001 to 30% by weight, more preferably 0.05-20% by weight, particularly preferably 1-10% by weight, based on the total weight of the preparation.

Prophylaxis or cosmetic or dermatological treatment with cosmetic or topical dermatological preparations having an effective content of N-(4-amino-2-methylquinolin-6-yl)-2-((4-ethylphenoxy)methyl)benzamide is carried out in the usual manner and specifically such that the cosmetic or topical dermatological preparations having an effective content of N-(4-amino-2-methylquinolin-6-yl)-2-((4-ethylphenoxy)methyl)benzamide are applied to the affected areas of skin.

N-(4-Amino-2-methylquinolin-6-yl)-2-((4-ethylphenoxy)methyl)benzamide may advantageously be incorporated into typical cosmetic and dermatological formulations, which can take different forms. Thus, they may for example be a solution, a water-in-oil (W/O) type emulsion or an oil-in-water (O/W) type emulsion, or multiple emulsions, for example a water-in-oil-in-water (W/O/W) type or oil-in-water-in-oil (O/W/O) type, a hydrodispersion or lipodispersion, a gel, a solid stick or even an aerosol.

Inventive emulsions in the context of the present invention, for example in the form of a cream, a lotion or a cosmetic milk, are advantageous and contain, for example, fats, oils, waxes, and/or other fatty substances, and also water and one or more emulsifiers as are customarily used for formulations of this type.

The water phase of the preparations according to the invention may advantageously contain customary cosmetic auxiliaries such as alcohols, in particular those having a low number of carbon atoms, preferably ethanol.

It also possible and advantageous in the context of the present invention to include the active substance used according to the invention in aqueous systems or in surfactant preparations for cleansing the skin and the hair.

It is of course known to those skilled in the art that sophisticated cosmetic compositions are generally not conceivable without the customary auxiliaries and additives. The cosmetic preparations according to the invention may therefore contain cosmetic auxiliaries as are typically used in such preparations, for example preservatives, bactericides, deodorant substances, antiperspirants, insect repellents, vitamins, antifoaming agents, dyes, pigments with a coloring effect, thickeners, softening substances, moisturizing and/or moisture-retaining substances, fats, oils, waxes or others customary components of a cosmetic formulation, such as alcohols, polyols, polymers, foam stabilizers, electrolytes, organic solvents or silicone derivatives.

Mutatis mutandis, corresponding requirements apply to the formulation of medicinal products.

Topical medicinal compositions in the context of the present invention generally comprise one or more medicaments at an effective concentration. For the sake of simplicity and in order to make a clear distinction between cosmetic and medicinal use and corresponding products, reference is made to the statutory provisions of the Federal Republic of Germany (for example the Kosmetikverordnung [Cosmetics Ordinance] and Lebensmittel- and Arzneimittelgesetz [Food and Medicinal Products Act]).

The oil phase of the emulsions of the present invention is advantageously selected from the group consisting of esters of saturated and/or unsaturated, branched and/or unbranched alkanecarboxylic acids having a chain length of 3 to 30 C atoms and saturated and/or unsaturated, branched and/or unbranched alcohols having a chain length of 3 to 30 C atoms, from the group consisting of esters of aromatic carboxylic acids and saturated and/or unsaturated, branched and/or unbranched alcohols having a chain length of 3 to 30 C atoms. Such ester oils may then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate, and also synthetic, semisynthetic, and natural mixtures of such esters, for example jojoba oil.

In addition, the oil phase may advantageously be selected from the group of the branched and unbranched hydrocarbons and hydrocarbon waxes, silicone oils, dialkyl ethers, the group consisting of saturated or unsaturated, branched or unbranched alcohols, and also fatty acid triglycerides, namely triglyceryl esters of saturated and/or unsaturated, branched and/or unbranched alkanecarboxylic acids having a chain length of 8 to 24, especially 12-18 C atoms. The fatty acid triglycerides may for example advantageously be selected from the group of the synthetic, semisynthetic, and natural oils, for example olive oil, sunflower oil, soybean oil, groundnut oil, rapeseed oil, almond oil, palm oil, coconut oil, palm kernel oil, argan oil, macadamia oil, olus oil, shea butter, cocoa butter, and other similar oils.

Any blends of such oil and wax components may also advantageously be used in the context of the present invention. It may optionally also be advantageous to use waxes, for example cetyl palmitate, as the sole lipid component of the oil phase.

Preparations that in accordance with the invention take the form of an emulsion particularly advantageously contain one or more hydrocolloids. These hydrocolloids may advantageously be selected from the group of the gums, polysaccharides, cellulose derivatives, phyllosilicates, polyacrylates, carbomers, Acrylates/C10-30 Alkyl Acrylate Crosspolymer, and/or other polymers.

Preparations that in accordance with the invention take the form of a hydrogel contain one or more hydrocolloids. These hydrocolloids may advantageously be selected from the abovementioned group.

### Efficacy Tests:

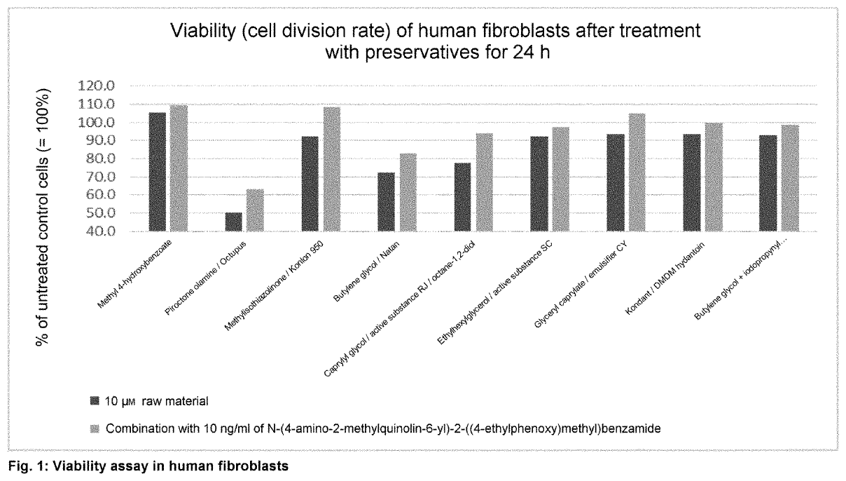

For the viability assay, dermal fibroblasts were treated with active substances/active substance combinations in the cell culture medium for 24 h and during this time were provided with BrdU (5-bromodeoxyuridine). The cells take up this synthetic nucleic acid, which is detectable by fluorescence detection, and incorporate it into newly synthesized genetic material during cell division. The fluorescence of the cell monolayer is then measured after 24 h, the intensity of which is ultimately a measure of the cell division rate during treatment with the active substance. This allows conclusions to be drawn about the effect of the individual active substances on cell viability. A positive effect of N-(4-amino-2-methylquinolin-6-yl)-2-((4-ethylphenoxy)methyl)benzamide in combination with a preservative is demonstrated by the increase in the rate of incorporation of BrdU versus treatment with the corresponding preservative alone, see FIG. 1.

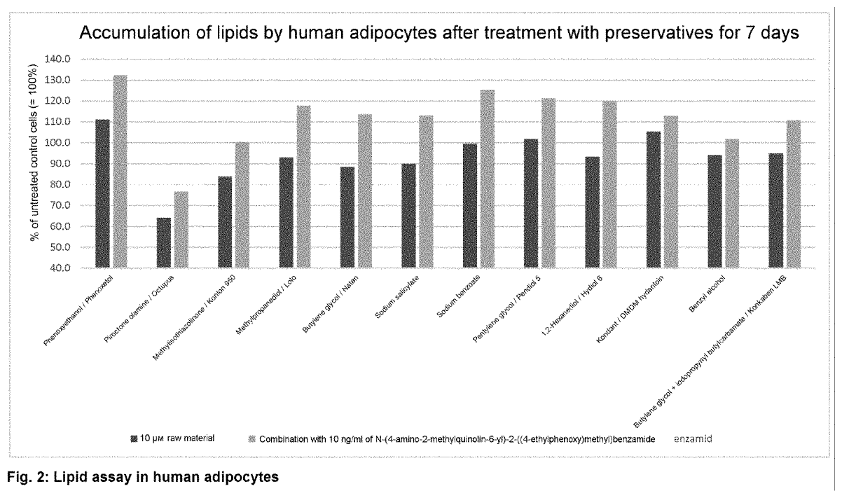

The lipid assay is performed on subcutaneous adipocytes. Cell culture dishes are inoculated with preadipocytes, which undergo differentiation into adipocytes. This is followed by treatment with the active substance for one week, during which the cells synthesize lipids and store them in vesicles. The total amount of lipid as a function of active substance effects is determined at the end of the culture period. This is done by staining the cell monolayer with the lipophilic fluorescent dye AdipoRed, followed by quantification by fluorescence spectroscopy. The positive effect of N-(4-amino-2-methylquinolin-6-yl)-2-((4-ethylphenoxy)methyl)benzamide in combination with a preservative is demonstrated by the increase in accumulation of AdipoRed and inferred increase in the differentiation performance of the adipocytes versus treatment with the corresponding preservative alone, see FIG. 2.

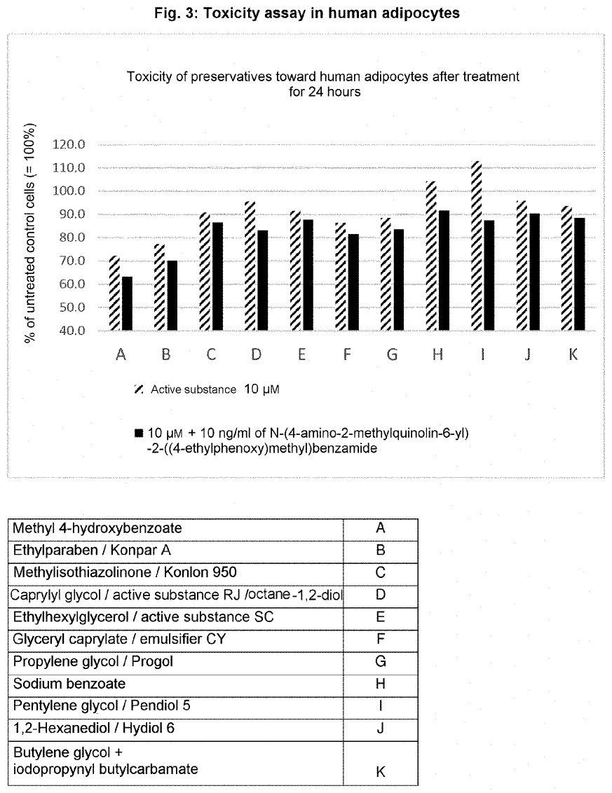

The third assay performed is the LDH (lactate dehydrogenase) cytotoxicity assay. In this assay, the metabolic enzyme LDH is detected in the medium supernatant of the cell monolayer by adding a substrate for the enzyme to the cell culture supernatant. Reaction of the latter produces a color change, which is determined photometrically. The magnitude of the color change correlates here with the amount of LDH in the cell culture supernatant. LDH is however an intracellular enzyme and is able to pass into the medium supernatant of cell cultures only if a cell is damaged, for example as a consequence of toxic substances, and if there is accompanying damage to the cell membrane. The positive effect of N-(4-amino-2-methylquinolin-6-yl)-2-((4-ethylphenoxy)methyl)benzamide in combination with a preservative is demonstrated by the decrease in the rate of escape of LDH versus treatment with the corresponding preservative alone, which equates to a reduction in the toxicity of the active-substance combination. See FIG. 3 for human adipocytes:

For all assays, the active substances were present as 1000× stocks dissolved to 10 mM in DMSO and were diluted 1:1000 in the appropriate cell culture medium for use.

### Tabulated Summary of the Results:

For the following active substances according to the invention, a tested combination together with 10 ng/ml of N-(4-amino-2-methylquinolin-6-yl)-2-((4-ethylphenoxy)methyl)benzamide, showed better activity in one or more cell culture assays than was observed for the corresponding active substance alone:

| Active substance | Assay |
|---|---|
| Methyl 4-hydroxybenzoate/Konpar M | LDH |
| Ethylparaben/Konpar A | BrdU + LDH |
| Phenoxyethanol/Phenoxetol | Lipid |
| Piroctone olamine/Octupus | Lipid + BrdU |
| Methylisothiazolinone/Konlon 950 | Lipid + BrdU |
| Methylpropanediol/Lolo | Lipid |
| Butylene glycol/Natan | Lipid + BrdU |
| Caprylyl glycol/active substance RJ/octane-1,2- | BrdU + LDH |
| diol | |
| Ethylhexylglycerol/active substance SC | BrdU + LDH |
| Glyceryl caprylate/emulsifier CY | BrdU + LDH |
| Propylene glycol/Progol | LDH |
| Sodium salicylate | Lipid |
| Sodium benzoate | Lipid + LDH |
| Pentylene glycol/Pendiol 5 | Lipid + LDH |
| 1,2-Hexanediol/Hydiol 6 | Lipid + LDH |
| Kondant/DMDM hydantoin | Lipid + BrdU |
| Benzyl alcohol | Lipid |
| Butylene glycol + iodopropynyl butylcarbamate/ | Lipid + BrdU + LDH |
| Konkaben LMB | |

### EXAMPLES

The examples below are intended to illustrate the present invention without limiting it. Unless otherwise stated, all quantitative data, fractions, and percentages are based on the weight and the total amount or on the total weight of the preparations.

| | A | B | C | D |
|---|---|---|---|---|
| | m | m | m | m |
| Composition in % | [%] | [%] | [%] | [%] |
| N-(4-Amino-2-methylquinolin-6-yl)-2- | 0.2 | 0.3 | 0.5 | 0.1 |
| ((4-ethylphenoxy)methyl) benzamide | | | | |
| Phenoxyethanol | 0.7 | 0.4 | 0.5 | 0.4 |
| Ethylhexylglycerol | | 0.5 | 0.25 | |
| | | | — | |
| Carnitine | | | 1 | |
| Creatine | | | 2 | |
| Creatinine | | | 0.1 | |
| Coenzyme Q10 | | | 0.5 | |
| Arctium lappa fruit extract | | | | 0.2 |
| Tocopherol | | | | 0.1 |
| Dicaprylyl ether | 3.5 | | | |
| Dimethicone | 0.5 | | | |
| Isopropyl palmitate | 2 | 9 | | |
| Cera Microcristallina | | 3 | | |
| Paraffinum Liquidum | | 3 | | |
| Isohexadecane | | 2 | | |
| Almond oil | | 5 | | |
| Shea butter | | | 3 | |
| C12-15 alkyl benzoates | | | 1 | |
| Caprylic/capric triglycerides | | | | 2 |
| Hydrogenated vegetable oil | | | | 3 |
| Octyldodecanol | | | | 2 |
| Argan oil | | | | 2 |
| Glyceryl stearate SE | 2 | | | |
| Sodium cetearyl sulfate | 0.3 | | | |
| Stearic acid | | 2.5 | | |
| Sodium stearoyl glutamate | | | 0.5 | |
| Glyceryl stearate | | | 2 | 1 |
| Glyceryl stearate citrate | | | | 1.5 |
| Tapioca starch | | | 1 | |
| Glycerol | 5 | 20 | 10 | 15 |
| Acrylates/C10-30 Alkyl Acrylate | | 0.3 | | 0.2 |
| Crosspolymer | | | | |
| Aloe vera | | 4 | | |
| Panthenol | | | | 1 |
| Urea | | | | 5 |
| Cetearyl alcohol | 1 | | | |
| Carbomer | 0.2 | | 0.5 | 0.1 |
| Xanthan gum | | 0.1 | | 0.2 |
| Alcohol denat | 2 | | | |
| Calcium chloride | | | 0.1 | |
| Sodium hydroxide, as required for | pH | pH | pH | pH |
| pH adjustment | 7.0 | 6.5 | 6.5 | 5.5 |
| Water | to | to | to | to |
| | 100 | 100 | 100 | 100 |
| | — | — | | — |

| | Example formulation | | | |
|---|---|---|---|---|
| | 1 | 2 | 3 | 4 |
| Chemical/INCI name | % by weight | % by weight | % by weight | % by weight |
| N-(4-Amino-2-methylquinolin-6-yl)-2- | 0.2 | 1 | 0.5 | 0.15 |
| ((4-ethylphenoxy)methyl)benzamide | | | | |
| Stearic acid | 2.50 | 2.00 | 2.00 | 2.50 |
| Glyceryl stearate | 1.00 | 1.00 | 1.00 | 1.00 |
| C12-15 alkyl benzoate | | | 3.00 | 2.00 |
| Caprylic acid/capric acid triglyceride | 2.50 | 2.50 | 2.00 | 2.50 |
| Isopropyl palmitate | 2.00 | | | 2.00 |
| Cetyl stearyl alcohol | 3.00 | | 2.00 | 3.00 |
| Cetyl alcohol | | 2.00 | | |
| Stearyl alcohol | | 2.00 | 1.00 | |
| C13-16 isoparaffin | | | | 1.00 |
| Dibutyl adioate | | | 1.50 | |
| Cyclomethicone | 1.00 | 1.00 | 0.50 | |
| Dicaprylyl carbonate | 2.00 | 2.00 | 2.00 | 2.00 |
| Dimethicone | 1.00 | — | 0.50 | 1.00 |
| Glycerol | 5.00 | 7.00 | 5.00 | 9.00 |
| Ethylhexyl cocoate | | | 1.00 | |
| Methylparaben | 0.20 | | | |
| Phenoxyethanol | 0.40 | 0.50 | 0.50 | 0.40 |
| Propylparaben | 0.10 | | | 0.10 |
| 1,2-Hexanediol | | | 0.10 | 0.10 |
| Ethylhexylglycerol | | | 0.20 | |
| Methylisothiazolinone | | 0.05 | | |
| Butylene glycol | | | 2.0 | |
| Carbomer | 0.15 | 0.10 | 0.15 | 0.10 |
| Carrageenan | 0.10 | | 0.10 | |
| Xanthan gum | | | 0.10 | |
| Acrylates/C10-30 Alkyl Acrylate | | 0.10 | | 0.10 |
| Crosspolymer | | | | |
| Trisodium EDTA | 0.20 | 0.20 | 0.20 | 0.20 |
| Tapioca starch | 1.50 | 1.00 | | |
| Polymethylsilsesquioxane | | 1.00 | 1.00 | |
| Aluminum starch octenylsuccinate | | | 1.00 | |
| Distarch phosphate | 1.00 | 1.00 | | 1.00 |
| Butyl Methoxydibenzoylmethane | 4 | 3 | | |
| Phenylbenzimidazole sulfonic acid | | 1 | | |
| Octocrylene | | 3 | | |
| Ethylhexyl salicylate | 4 | | | |
| Sodium benzoate | 0.01 | 0.05 | 0.10 | 0.15 |
| Hydroxypropyl tetrahydropyrantriol | 1.00 | 0.50 | | |
| Lipoic acid | | 0.50 | 0.20 | |
| Vitamin B6 HCl | 0.10 | 0.05 | | 0.30 |
| Tranexamic acid | | 0.01 | 0.25 | |
| Pyrus malus stem extract | 1.00 | 0.25 | 0.50 | 0.75 |
| Sodium hydroxide | as | as | as | as |
| | required | required | required | required |
| Parfum | 0.30 | | 0.20 | 0.20 |
| Sodium hydroxide | as | as | as | as |
| | required | required | required | required |
| Water | to 100 | to 100 | to 100 | to 100 |

Example formulation

| | 5 | 6 | 7 | 8 |
|---|---|---|---|---|
| Chemical name | % by weight | % by weight | % by weight | % by weight |
| N-(4-Amino-2-methylquinolin-6-yl)-2- | 0.1 | 0.3 | 0.2 | 0.2 |
| ((4-ethylphenoxy)methyl)benzamide | | | | |
| Glyceryl stearate citrate | 2.00 | 1.50 | 2.00 | 2.00 |
| Behenyl alcohol | 1.50 | | | |
| C12-15 alkyl benzoate | 2.00 | 2.50 | 2.00 | 2.50 |
| Caprylic acid/capric acid triglyceride | 2.00 | 2.00 | 2.50 | 2.50 |
| Cetyl alcohol | 2.00 | 2.00 | | 2.00 |
| Cetyl stearyl alcohol | | | 2.00 | |
| Cyclopentasiloxane | | | | 1.00 |
| Cyclomethicone | 1.00 | 1.00 | 2.00 | 2.00 |
| Dicaprylyl carbonate | | 2.00 | 2.50 | 2.50 |
| Paraffinum Liquidum (mineral oil) | | | 0.50 | |
| Octyldodecanol | | 2.00 | | |
| Isopropyl palmitate | 1.50 | | | |
| Dimethicone | 0.50 | 1.00 | 1.00 | |
| Glycerol | 3.00 | 5.00 | 7.00 | 9.00 |
| Methylparaben | 0.20 | 0.15 | | |
| Phenoxyethanol | 0.40 | 0.60 | 0.50 | 0.50 |
| Propylparaben | 0.10 | | | |
| Methylisothiazolinone | | | 0.05 | |
| Piroctone olamine | | | | 0.15 |
| Sodium benzoate | 0.10 | 0.10 | 0.10 | 0.10 |
| Glyceryl caprylate | | | | 0.20 |
| Ethylparaben | 0.10 | | | |
| Carbomer | 0.20 | | 0.15 | 0.15 |
| Sodium polyacrylate | | 0.40 | | |
| Xanthan gum | 0.10 | | 0.10 | |
| Acrylates/C10-30 Alkyl Acrylate | | 0.10 | | 0.10 |
| Crosspolymer | | | | |
| Tapioca starch | 0.50 | | 0.50 | |
| Nylon-12 (1,8-diazacyclotetradecane- | 1.00 | | | 1.00 |
| 2,7-dione homopolymer) | | | | |
| Polymethylsilsesquioxane | | 1.00 | | |
| Aluminum starch octenylsuccinate | | 1.00 | | |
| Glycyrrhiza inflata root extract | 0.03 | 0.05 | 0.05 | 0.03 |
| Titanium dioxide | | 1.00 | | |
| Octocrylene | 1.00 | 2.00 | 1.00 | 1.00 |
| Bis-Ethylhexyloxyphenol | 1.00 | | | |
| Methoxyphenyl Triazine | | | | |
| Sodium hydroxide | as | as | as | as |
| | required | required | required | required |
| Trisodium EDTA | 0.15 | | 0.15 | |
| 1-(1,2,3,4,5,6,7,8-Octahydro-2,3,8,8- | 0.1 | | as | as |
| tetramethyl-2-naphthyl)ethan-1-one | | | required | required |
| Geraniol | | 0.05 | | |
| Hexylcinnamaldehyde | | | 0.05 | |
| Sodium hydroxide | as | as | as | as |
| | required | required | required | required |
| Parfum | 0.10 | 0.20 | 0.30 | |
| Water | to 100 | to 100 | to 100 | to 100 |

| | Example formulations | | | |
|---|---|---|---|---|
| | 9 | 10 | 11 | 12 |
| Chemical name | % by weight | % by weight | % by weight | % by weight |
| N-(4-Amino-2-methylquinolin-6-yl)-2- | 0.3 | 0.2 | 0.3 | 0.1 |
| ((4-ethylphenoxy)methyl)benzamide | | | | |
| Polyglyceryl-3 methylglucose | 2.00 | 2.50 | 2.50 | 2.50 |
| distearate | | | | |
| Sorbitan stearate | 1.50 | 3.00 | 1.50 | 3.00 |
| C12-15 alkyl benzoate | 2.50 | | 2.50 | 2.50 |
| Caprylic acid/capric acid triglycerides | 2.50 | 2.50 | 2.50 | |
| Stearyl alcohol | 1.00 | 1.50 | 1.00 | 1.50 |
| Cyclomethicone | | 1.00 | 2.00 | 1.00 |
| Isopropyl myristate | | 2.50 | 2.00 | 2.50 |
| Isopropyl palmitate | 2.00 | | 1.00 | |
| Ethylhexyl stearate | | 1.50 | | |
| Dimethicone | | 1.00 | | 1.00 |
| Decyl oleate | | — | 1.50 | |
| Glycerol | 5.00 | 7.50 | 3.00 | 7.50 |
| Butyrospermum parkii butter | 2.00 | | | |
| Squalane | 0.50 | | | |
| Methylparaben | 0.20 | 0.20 | | |
| Phenoxyethanol | 0.40 | | 0.40 | 0.40 |
| Propylparaben | 0.10 | | | |
| Caprylyl glycol | | 0.20 | | |
| Ethylhexylglycerol | | 0.20 | | 0.2 |
| Pentylene glycol | | 0.10 | 0.05 | 0.5 |
| Carbomer | 0.15 | 0.10 | 0.15 | 0.10 |
| Ammonium Acryloyldimethyltaurate/VP | | 0.20 | | 0.20 |
| Copolymer | | | | |
| Carrageenan | 0.10 | | 0.15 | |
| Trisodium EDTA | | 1.00 | | 1.00 |
| Tapioca starch | | 1.00 | 1.00 | |
| Distarch phosphate | | 1.00 | | 1.00 |
| Acrylonitrile-methacrylonitrile-methyl | | | 1.00 | 1.00 |
| methacrylate copolymer + isopentane + | | | | |
| magnesium hydroxide | | | | |
| Diethylamino Hydroxybenzoyl Hexyl | 1.00 | 2.00 | 1.00 | 1.00 |
| Benzoate | | | | |
| Ethylhexyl methoxycinnamate | 1.00 | 1.00 | 2.00 | 2.00 |
| Butyl Methoxydibenzoylmethane | 2.00 | 2.00 | 1.00 | 2.00 |
| Octocrylene | 1.00 | 1.00 | 2.00 | 1.00 |
| Titanium dioxide | | | 1.00 | |
| Sodium hydroxide | as | as | as | as |
| | required | required | required | required |
| Ubiquinone | 0.10 | | | |
| Sodium metabisulfite | | 0.15 | | |
| BHT (tert-butylhydroxytoluene) | | | 0.05 | |
| Sodium hydroxide | as | as | as | as |
| | required | required | required | required |
| Parfum | | | as | as |
| | | | required | required |
| Water | to 100 | to 100 | to 100 | to 100 |

| | Example formulations | | | |
|---|---|---|---|---|
| | 13 | 14 | 15 | 16 |
| Chemical name | % by weight | % by weight | % by weight | % by weight |
| N-(4-Amino-2-methylquinolin-6-yl)-2-((4- | 0.6 | 1.2 | 2 | 0.1 |
| ethylphenoxy)methyl)benzamide | | | | |
| PEG-40 stearate | 0.80 | 1.00 | 1.00 | 1.00 |
| Glyceryl stearate | 2.50 | 3.00 | | 3.00 |
| C12-15 alkyl benzoate | 2.00 | 2.50 | 2.00 | 2.00 |
| Caprylic acid/capric acid triglyceride | 2.00 | | 2.50 | 2.00 |
| Cetyl stearyl alcohol | 3.00 | 3.00 | 3.00 | 3.00 |
| Cyclomethicone | 2.00 | | | 2.00 |
| Dicaprylyl carbonate | | 2.00 | 2.50 | 2.50 |
| Octyldodecanol | 1.00 | | | 1.50 |
| Triisostearin | | 0.50 | | 1.00 |
| Butyrospermum parkii butter | 2.00 | | | |
| Octyldodecyl myristate | 1.00 | | | 1.00 |
| Dimethicone | 1.00 | 1.00 | 1.00 | 1.00 |
| Glycerol | 7.50 | 5.00 | 9.0 | 7.50 |
| Methylparaben | 0.20 | | 0.10 | — |
| Phenoxyethanol | 0.40 | 0.50 | 0.40 | 0.40 |
| Propylparaben | 0.10 | | | |
| Glyceryl caprylate | | 0.25 | | |
| Pentylene glycol | | 0.50 | | |
| Butylene glycol | | | 3.00 | |
| Lauroyl ethyl arginate | 0.10 | 0.05 | 0.20 | |
| Potassium sorbate | 0.10 | 0.05 | 0.15 | 0.01 |
| Sodium salicylate | 0.01 | 0.02 | 0.01 | 0.05 |
| Carbomer | 0.15 | 0.10 | 0.10 | 0.15 |
| Sodium polyacrylate | | 0.20 | 0.20 | |
| Xanthan gum | 0.10 | | | |
| Acrylates/C10-30 Alkyl Acrylate | | | | 0.1 |
| Crosspolymer | | | | |
| Trisodium EDTA + water (20% aqueous | | 1.00 | 1.00 | 1.00 |
| solution) | | | | |
| Tapioca starch | | 1.00 | 1.00 | 1.00 |
| Distarch phosphate | | 1.00 | 1.00 | 1.00 |
| Aluminum starch octenylsuccinate | 2.00 | | | |
| N-(4-(2,4-Dihydroxyphenyl)thiazol-2- | 0.10 | 0.15 | 0.10 | 0.01 |
| yl)isobutyramide | | | | |
| Ethylhexyl methoxycinnamate | 1.00 | | | |
| Diethylamino Hydroxybenzoyl Hexyl | 0.50 | 1.00 | 2.00 | 1.00 |
| Benzoate | | | | |
| Homosalate (3,3,5-trimethylcyclohexyl | 2.00 | 2.00 | 1.00 | 2.00 |
| salicylate) | | | | |
| Phenylbenzimidazole sulfonic acid | 1.00 | 1.00 | 2.00 | 1.00 |
| Titanium dioxide | | | 1.00 | |
| Glyceryl glucoside | 3.00 | | | |
| Short-chain hyaluronic acid | | 0.10 | | |
| Long-chain hyaluronic acid | | | 0.10 | |
| 4-Butylresorcinol | | | | 0.30 |
| Magnolia bark extract | 0.10 | | | |
| Octadecenedioic acid | | 0.05 | | |
| Folic acid | | | 0.01 | |
| Carnitine | | | | 0.50 |
| Creatine | 0.10 | | | |
| Alpha-Glucosylrutin | | 0.01 | | |
| Taurine | | | 0.10 | |
| Mulberry root extract | | | | 0.20 |
| Sodium metabisulfite | 0.10 | | | |
| Diethylhexyl syringylidenemalonate | 0.13 | 0.13 | | |
| Sodium hydroxide | as | as | as | as |
| | required | required | required | required |
| 3-Methyl-5-phenyl-1-pentanol | 0.10 | | | |
| Coumarin | | 0.05 | | |
| Ethyllinalool | | | 0.10 | |
| Ascorbyl palmitate | 0.10 | | | |
| Sodium hydroxide | as | as | as | as |
| | required | required | required | required |
| Parfum | as | as | as | |
| | required | required | required | |
| Water | to 100 | to 100 | to 100 | to 100 |

| | Example formulations | | | |
|---|---|---|---|---|
| | 17 | 18 | 19 | 20 |
| Chemical name | % by weight | % by weight | % by weight | % by weight |
| N-(4-Amino-2-methylquinolin-6-yl)-2- | 0.2 | 0.3 | 0.5 | 1 |
| ((4-ethylphenoxy)methyl)benzamide | | | | |
| Glyceryl stearate citrate | 2.00 | 2.00 | 2.00 | 2.00 |
| Isopropyl palmitate | 3.00 | 2.00 | 3.00 | 1.00 |
| Cetyl stearyl alcohol | 4.00 | 3.00 | 3.00 | |
| Cetyl alcohol | | | | 4.00 |
| Caprylic acid/capric acid triglyceride | 3.00 | 2.50 | 2.00 | 3.00 |
| C12-15 alkyl benzoate | 3.00 | 2.50 | 2.00 | 2.00 |
| Cyclomethicone | 1.00 | | 1.00 | |
| Dicaprylyl carbonate | | | 2.50 | |
| Dimethicone | | 0.50 | | |
| Octyldodecyl myristate | | 1.00 | | |
| Glycerol | 4.00 | 6.00 | 5.00 | 6.00 |
| Methylparaben | 0.20 | | 0.10 | |
| Phenoxyethanol | 0.40 | 0.40 | 0.40 | 0.40 |
| Piroctone olamine | | | | 0.10 |
| Ethylhexylglycerol | | 0.30 | | |
| Glyceryl caprylate | | 0.30 | | |
| 2-Methyl-1,3-propanediol | | 2.00 | | 2.00 |
| Sodium benzoate | 0.2 | | 0.20 | 0.10 |
| Carbomer | 0.20 | 0.10 | 0.15 | |
| Sodium polyacrylate | | 0.40 | | |
| Xanthan gum | 0.10 | | | 0.15 |
| Acrylates/C10-30 Alkyl Acrylate | | | 0.10 | 0.20 |
| Crosspolymer | | | | |
| Acrylonitrile-methacrylonitrile-methyl | 0.50 | | 0.50 | |
| methacrylate copolymer + isopentane + | | | | |
| magnesium hydroxide | | | | |
| Aluminum starch octenylsuccinate | | 1.00 | | 1.00 |
| Methyl Methacrylate Crosspolymer | 1.00 | | | 1.00 |
| Glycyrrhiza inflata root extract | 0.03 | | | |
| Vitamin C/ascorbic acid | | 3.00 | | |
| Glycine Soja Germ Extract | | | 0.50 | |
| Arctium lappa root extract | | | | 0.30 |
| Pimpinella anisum fruit extract | 4.00 | | | |
| Glycyrrhetinic acid | | 0.10 | | |
| N-Acetylhydroxyproline | | | 0.10 | |
| Niacinamide | | | | 0.20 |
| Magnesium ascorbyl phosphate | 0.10 | | | |
| Ellagic acid | | 0.01 | | |
| Licorice root extract | | | 0.10 | |
| Sea salt | | | | 0.05 |
| Isoserinol | 1.00 | | | |
| N-(4-(2,4-Dihydroxyphenyl)thiazol-2- | 0.25 | 0.30 | 0.01 | 0.05 |
| yl)isobutyramide | | | | |
| Bis-Ethylhexyloxyphenol Methoxyphenyl | 1.00 | | 1.00 | 1.00 |
| Triazine | | | | |
| Octocrylene | 1.00 | | 2.00 | 2.00 |
| Butyl Methoxydibenzoylmethane | 2.00 | | 1.00 | 2.00 |
| Ethylhexyl salicylate | 1.00 | | 2.00 | 1.00 |
| Sodium hydroxide | as | as | as | as |
| | required | required | required | required |
| Water | to 100 | to 100 | to 100 | to 100 |

| | Example formulations | | | |
|---|---|---|---|---|
| | 21 | 22 | 23 | 24 |
| Chemical name | % by weight | % by weight | % by weight | % by weight |
| N-(4-Amino-2-methylquinolin-6-yl)-2-((4- | | | | |
| ethylphenoxy)methyl) benzamide | | | | |
| Sucrose polystearate + hydrogenated | 1.00 | 1.00 | | |
| polyisobutene | | | | |
| Sodium stearoyl glutamate | 0.20 | 0.20 | 0.30 | 0.30 |
| C12-15 alkyl benzoate | 1.50 | 1.50 | | |
| Cetyl alcohol | 0.50 | 0.50 | | |
| Glycerol | 7.50 | 7.50 | 5.00 | 5.00 |
| Isopropyl stearate | 1.00 | 1.00 | 2.00 | 2.00 |
| Paraffinum Liquidum (mineral oil) | 3.00 | 3.00 | 1.00 | 1.00 |
| Methylparaben | 0.10 | | | 0.10 |
| Ethylhexylglycerol | | | 0.30 | 0.10 |
| Propylparaben | 0.10 | | | |
| Phenoxyethanol | 0.40 | 0.50 | 0.40 | 0.40 |
| Ascorbyl glucoside | 0.10 | | | |
| Undecenoylphenylalanine | | 0.50 | | |
| Kojic acid | | | 0.10 | |
| Arbutin | | | | 0.01 |
| Betaine | 0.20 | | | |
| Ethylhexyl methoxycinnamate | | 2.00 | 1.00 | 1.00 |
| Butyl Methoxydibenzoylmethane | | 1.00 | 2.00 | 2.00 |
| Phenylbenzimidazole sulfonic acid | | 2.00 | 1.00 | 2.00 |
| Acrylates/octylacrylamide copolymer | | 1.00 | | |
| Butylene glycol | | | 3.00 | |
| Polymethylsilsesquioxane | | | 1.00 | 1.00 |
| Prunus amygdalus dulcis oil | | | 1.00 | |
| Nylon-12 (1,8-diazacyclotetradecane-2,7- | | 1.00 | 1.00 | |
| dione homopolymer) | | | | |
| Distarch phosphate | | 1.00 | | 1.00 |
| Methyl Methacrylate Crosspolymer | 1.00 | | | |
| Aluminum starch octenylsuccinate | 1.00 | | | |
| Ammonium Acryloyldimethyltaurate/VP | | | 0.25 | 0.25 |
| Copolymer | | | | |
| Xanthan gum | 0.10 | | | 0.10 |
| Acrylates/C10-30 Alkyl Acrylate | 0.25 | 0.10 | | |
| Crosspolymer | | | | |
| Carbomer | | 0.10 | 0.10 | |
| Parfum | | 0.20 | | 0.20 |
| Sodium hydroxide | as | as | as | as |
| | required | required | required | required |
| Water | to 100 | to 100 | to 100 | to 100 |

| | Example formulations | | | |
|---|---|---|---|---|
| | 25 | 26 | 27 | 28 |
| Chemical name | % by weight | % by weight | % by weight | % by weight |
| N-(4-Amino-2-methylquinolin-6-yl)-2-((4- | 0.2 | 0.3 | 1 | 0.5 |
| ethylphenoxy)methyl)benzamide | | | | |
| Sodium cetearyl sulfate | 0.15 | 0.15 | | 0.15 |
| Glyceryl stearate SE | 2.00 | 2.00 | | 1.50 |
| Sodium stearoyl glutamate | | | 0.30 | |
| C12-15 alkyl benzoate | 2.50 | 2.50 | 2.50 | 2.50 |
| Octyldodecanol | 1.00 | 1.00 | | |
| Caprylic acid/capric acid triglyceride | 2.00 | 2.00 | 2.00 | 2.00 |
| Cetyl stearyl alcohol | 2.00 | 2.00 | 3.00 | 1.00 |
| Glyceryl stearate | | | 2.00 | |
| Glycerol | 5.00 | 5.00 | 7.50 | 7.50 |
| Cetearyl alcohol | 1.00 | 1.50 | 1.00 | 1.00 |
| Isopropyl stearate | 3.00 | 3.00 | 2.00 | 2.00 |
| Paraffinum Liquidum (mineral oil) | 2.00 | 2.00 | 1.00 | 1.00 |
| Methylisothiazolinone | | | | 0.05 |
| Phenoxyethanol | 0.40 | 0.50 | 0.40 | 0.30 |
| Methylparaben | 0.15 | | | |
| Propylparaben | 0.10 | | | |
| Piroctone olamine | | 0.15 | | |
| Ethylhexylglycerol | | | 0.4 | |
| Ethylhexyl methoxycinnamate | 1.00 | 2.00 | 1.00 | 1.00 |
| Butyl Methoxydibenzoylmethane | 1.00 | 1.00 | 2.00 | 2.00 |
| Pentylene glycol | — | 1.00 | 1.00 | |
| Butylene glycol | 1.00 | 1.50 | 3.00 | 3.00 |
| Dipropylene glycol | 0.50 | 1.00 | 0.80 | 0.10 |
| 1,2-Hexanediol | | | | 1.00 |
| Carbomer | | | 0.10 | 0.15 |
| Ammonium Acryloyldimethyltaurate/VP | 0.20 | | | |
| Copolymer | | | | |
| Chondrus crispus | 0.10 | 0.10 | | |
| Xanthan gum | | | 0.10 | |
| Acrylates/C10-30 Alkyl Acrylate Crosspolymer | | 0.20 | 0.10 | 0.10 |
| Parfum | 0.20 | | | 0.20 |
| Sodium hydroxide | as | as | as | as |
| | required | required | required | required |
| Water | to 100 | to 100 | to 100 | to 100 |

| | Example formulations | | | |
|---|---|---|---|---|
| | 29 | 30 | 31 | 32 |
| Chemical/INCI name | % by weight | % by weight | % by weight | % by weight |
| N-(4-Amino-2-methylquinolin-6-yl)-2-((4- | 0.4 | 0.1 | 0.2 | 0.3 |
| ethylphenoxy)methyl)benzamide | | | | |
| Sodium cetearyl sulfate | 0.15 | 0.15 | 0.20 | 0.20 |
| Glyceryl stearate, self-emulsifying | 2.00 | 2.00 | 1.50 | 1.50 |
| C12-15 alkyl benzoate | 2.00 | 2.00 | 2.00 | 2.00 |
| Octyldodecanol | 1.00 | 1.00 | — | — |
| Caprylic acid/capric acid triglyceride | 2.00 | 2.00 | 2.00 | 2.00 |
| Cetyl stearyl alcohol | 2.00 | 2.00 | 1.00 | 1.00 |
| Cyclomethicone | 1.00 | 1.00 | 2.00 | 2.00 |
| Dimethicone | 0.50 | 0.50 | 1.00 | 1.00 |
| Glycerol | 5.00 | 5.00 | 7.50 | 7.50 |
| Isopropyl palmitate | 2.50 | 2.50 | 2.00 | 2.00 |
| DMDM hydantoin | 0.05 | 0.05 | 0.05 | 0.05 |
| Phenoxyethanol | 0.35 | 0.25 | 0.30 | 0.30 |
| Ethanol | | | 3.00 | 2.00 |
| Pentylene glycol | 1.00 | | 1.00 | 1.50 |
| Potassium sorbate | 0.10 | 0.30 | | 0.05 |
| Zingerone | 0.10 | | | |
| Dihydromyricetin | | 0.03 | | |
| White tea extract | | | 1.00 | |
| 4-Hexylresorcinol | | | | 0.30 |
| Phenylethylresorcinol | 0.50 | | | |
| Ubiquinone | | 0.10 | | |
| Cyanomethylphenyl Menthane Carboxamide | | | 0.10 | |
| Menthoxypropanediol | | | | 0.10 |
| Menthane Carboxamide Ethylpyridine | 0.10 | | | |
| Hydroxyethylurea | | 0.50 | | |
| Urea | | | 1.00 | |
| N-(4-(2,4-Dihydroxyphenyl)thiazol-2- | 0.10 | 0.10 | 0.05 | 0.05 |
| yl)isobutyramide | | | | |
| Carbomer | 0.20 | 0.20 | 0.20 | 0.20 |
| Carrageenan | 0.10 | 0.10 | | |
| Xanthan gum | | | 0.20 | 0.20 |
| Acrylates/C10-30 Alkyl Acrylate Crosspolymer | | | | 0.15 |
| Sodium polyacrylate | | 0.20 | | |
| Diethylhexyl 2,6-naphthalate | | | 1.00 | |
| Phenylbenzimidazole sulfonic acid | 1.00 | 2.00 | 1.00 | 1.00 |
| Titanium dioxide | 1.00 | 1.00 | 2.00 | 2.00 |
| Diethylamino Hydroxybenzoyl Hexyl Benzoate | 2.00 | 2.00 | 1.00 | 2.00 |
| Octocrylene | 1.00 | 1.00 | 2.00 | 1.00 |
| 3,3,5-Trimethylcyclohexyl salicylate | | 1.00 | | |
| Distarch phosphate | | 1.00 | 1.00 | — |
| Methyl Methacrylate Crosspolymer | 1.00 | | | 1.00 |
| Polymethylsilsesquioxane | | | 1.00 | 1.00 |
| Acrylonitrile-methacrylonitrile-methyl | 1.00 | 1.00 | | |
| methacrylate copolymer + isopentane + | | | | |
| magnesium hydroxide | | | | |
| 1-(1,2,3,4,5,6,7,8-Octahydro-2,3,8,8- | | 0.10 | 0.10 | |
| tetramethyl-2-naphthyl)ethan-1-one | | | | |
| Hydroxyisohexyl 3-cyclohexenecarboxaldehyde | 0.05 | 0.05 | 0.10 | |
| Linalyl acetate | 0.10 | | 0.05 | |
| Parfum | 0.15 | 0.15 | 0.30 | |
| Sodium hydroxide | as | as | as | as |
| | required | required | required | required |
| Water | to 100 | to 100 | to 100 | to 100 |

| | Example formulations | | | |
|---|---|---|---|---|
| | 33 | 34 | 35 | 36 |
| Chemical/INCI name | % by weight | % by weight | % by weight | % by weight |
| N-(4-Amino-2-methylquinolin-6-yl)-2-((4- | 0.1 | 0.2 | 0.25 | 0.7 |
| ethylphenoxy)methyl)benzamide | | | | |
| Sodium cetearyl sulfate | 0.15 | 0.15 | 0.15 | 0.15 |
| Glyceryl stearate, self-emulsifying | 1.00 | 1.00 | 1.00 | 1.00 |
| C12-15 alkyl benzoate | 2.00 | 2.50 | 2.00 | 2.00 |
| Isopropyl palmitate | 3.50 | 3.00 | 2.50 | 3.50 |
| Dimethicone | | | | 0.5 |
| Cetyl stearyl alcohol | 1.00 | 1.00 | 1.00 | 1.00 |
| Octyldodecyl myristate | | | | 1.00 |
| Butyrospermum parkii butter | | | 1.00 | |
| Glycerol | 7.00 | 3.00 | 9.00 | 5.00 |
| Carbomer | 0.3 | | 0.10 | 0.10 |
| Acrylates/C10-30 Alkyl Acrylate Crosspolymer | 0.15 | 0.10 | | 0.3 |
| Xanthan gum | | 0.15 | 0.15 | 0.15 |
| Phenylbenzimidazole sulfonic acid | 1.00 | 1.00 | 0.50 | 1.00 |
| Butyl Methoxydibenzoylmethane | 1.50 | 1.50 | 1.50 | 1.50 |
| Ethylhexyl salicylate | 2.00 | 2.50 | 2.50 | 2.50 |
| Octocrylene | 1.50 | 1.50 | 2.50 | 1.50 |
| Titanium dioxide + trimethoxycaprylylsilane | 1.00 | | 1.00 | |
| Aluminum starch octenylsuccinate | | 1.00 | | 0.50 |
| Methyl Methacrylate Crosspolymer | 0.50 | | 0.50 | |
| Tapioca starch | 0.50 | 0.50 | | 1.00 |
| Phenoxyethanol | 0.50 | 0.50 | 0.50 | 0.40 |
| Ethylhexylglycerol | 0.25 | | 0.25 | |
| 1,2-Hexanediol | | 1.00 | | 3.00 |
| Caprylyl glycol | | 0.30 | 0.30 | |
| 2-Methyl-1,2-propanediol | 2.00 | 2.00 | 2.00 | |
| 1,3-Butanediol | 0.01 | | 0.20 | |
| Sodium benzoate | | 0.10 | | 0.20 |
| Sodium hydroxide | as | as | as | as |
| | required | required | required | required |
| Water | to 100 | to 100 | to 100 | to 100 |

| | Example formulations | | | |
|---|---|---|---|---|
| | 37 | 38 | 39 | 40 |
| Chemical/INCI name | % by weight | % by weight | % by weight | % by weight |
| N-(4-Amino-2-methylquinolin-6-yl)-2-((4- | 0.25 | 0.6 | 0.1 | 0.05 |
| ethylphenoxy)methyl)benzamide | | | | |
| Polyglyceryl-10 stearate | 0.20 | 0.20 | 0.20 | 0.20 |
| Glyceryl stearate | 3.00 | 0.50 | 0.50 | 0.50 |
| C12-15 alkyl benzoate | 4.00 | 2.00 | 1.50 | 2.50 |
| Isopropyl palmitate | 4.00 | 1.00 | 2.00 | 2.50 |
| Caprylic acid/capric acid triglyceride | 4.00 | 3.00 | 2.00 | 2.50 |
| Hydrogenated coco-glycerides | 3.00 | | | 2.00 |
| Butyrospermum parkii butter | 3.00 | | 2.50 | — |
| Cetyl stearyl alcohol | 5.00 | 3.50 | 4.00 | 3.00 |
| Paraffinum Liquidum (mineral oil) | | | | 1.00 |
| Glycerol | 5.00 | 3.00 | 7.00 | 9.00 |
| Acrylates/C10-30 Alkyl Acrylate Crosspolymer | 0.30 | 0.20 | 0.15 | 0.20 |
| Methylisothiazolinone | 0.05 | | | |
| Phenoxyethanol | 0.50 | 0.40 | 0.40 | 0.40 |
| Carbomer | 0.10 | 0.15 | 0.10 | 0.10 |
| Ethylparaben | | 0.10 | 0.10 | |
| Benzyl alcohol | | 0.10 | 0.05 | 0.15 |
| Polymethylsilsesquioxane | | 1.00 | 0.50 | |
| Methyl Methacrylate Crosspolymer | | | 1.00 | 0.50 |
| Tapioca starch | 0.50 | | | 0.50 |
| Benzophenone-4 | 1.00 | 2.00 | 1.50 | 0.50 |
| Ethylhexyl triazone | 2.00 | 0.50 | 1.00 | 2.00 |
| Ethanol | 3.00 | | 2.00 | |
| Geraniol | 0.05 | 0.05 | | |
| Benzyl salicylate | | 0.05 | 0.05 | |
| Ethyllinalool | | | 0.05 | 0.05 |
| Parfum | 0.20 | 0.15 | | 0.30 |
| Sodium hydroxide | as | as | as | as |
| | required | required | required | required |
| Water | to 100 | to 100 | to 100 | to 100 |

| | Example formulations | | | |
|---|---|---|---|---|
| | 41 | 42 | 43 | 44 |
| Chemical/INCI name | % by weight | % by weight | % by weight | % by weight |
| N-(4-Amino-2-methylquinolin-6-yl)-2-((4- | 0.2 | 0.1 | 0.5 | 1 |
| ethylphenoxy)methyl)benzamide | | | | |
| Polyglyceryl-10 stearate | 0.20 | 0.20 | 0.15 | 0.15 |
| C12-15 alkyl benzoate | 2.50 | 2.50 | 2.00 | 3.00 |
| Isopropyl palmitate | 2.50 | 2.50 | 2.00 | 2.00 |
| Caprylic acid/capric acid triglyceride | 2.00 | 2.50 | 1.00 | 2.00 |
| Glyceryl stearate | 1.00 | 1.00 | 0.50 | 0.50 |
| Octyldodecanol | 0.50 | | | 1.00 |
| Cyclomethicone | | | 0.50 | 0.50 |
| Butyl Methoxydibenzoylmethane | 1.00 | 2.00 | 2.00 | 1.00 |
| Octocrylene | 0.50 | 2.00 | 3.00 | 2.00 |
| Ethylhexyl salicylate | 2.00 | 1.00 | 1.00 | 1.50 |
| Phenylbenzimidazole sulfonic acid | 1.00 | 1.00 | 0.50 | 1.50 |
| Titanium dioxide | | 1.00 | | 1.00 |
| 3,3,5-Trimethylcyclohexyl salicylate | | | 1.00 | 1.00 |
| Glycerol | 9.00 | 5.00 | 7.00 | 7.00 |
| Tapioca starch | 1.00 | 1.00 | | |
| Acrylonitrile-methacrylonitrile-methyl | | 1.00 | 0.50 | |
| methacrylate copolymer + isopentane + | | | | |
| magnesium hydroxide | | | | |
| Distarch phosphate | | | | 1.00 |
| Phenoxyethanol | 0.50 | | 0.40 | |
| Ethylhexylglycerol | 0.2 | 0.5 | 0.10 | 0.5 |
| Benzyl alcohol | | 0.2 | | |
| Carbomer | 0.25 | 0.20 | 0.20 | 0.20 |
| Acrylates/C10-30 Alkyl Acrylate Crosspolymer | 0.20 | | | 0.15 |
| Ammonium Acryloyldimethyltaurate/VP | | 0.25 | | |
| Copolymer | | | | |
| Sodium polyacrylate | | | 0.30 | |
| Xanthan gum | | | | 0.15 |
| Ethanol | 3.00 | 3.00 | | |
| Butylene glycol | | | 2.00 | 2.00 |
| Coumarin | | 0.05 | 0.05 | |
| Hexylcinnamaldehyde | 0.05 | 0.05 | | 0.05 |
| Hexyl salicylate | | | 0.05 | 0.05 |
| Parfum | 0.15 | 0.20 | | 0.30 |
| Sodium hydroxide | as | as | as | as |
| | required | required | required | required |
| Water | to 100 | to 100 | to 100 | to 100 |

| | Example formulations | | | |
|---|---|---|---|---|
| | 45 | 46 | 47 | 48 |
| Chemical/INCI name | % by weight | % by weight | % by weight | % by weight |
| N-(4-Amino-2-methylquinolin-6-yl)-2-((4- | 0.2 | 0.1 | 0.05 | 0.5 |
| ethylphenoxy)methyl)benzamide | | | | |
| Potassium cetyl phosphate | 0.20 | 0.20 | 0.20 | 0.20 |
| Dicaprylyl carbonate | | 1.00 | | |
| C12-15 alkyl benzoate | 2.50 | 2.00 | 1.00 | 3.00 |
| Isopropyl palmitate | 2.50 | 2.00 | 3.00 | 1.00 |
| Caprylic acid/capric acid triglyceride | 2.50 | 2.00 | 1.50 | 2.00 |
| Cera Microcristallina | | | | 0.50 |
| Cyclomethicone | 0.25 | | 0.50 | 0.50 |
| Diethylhexyl 2,6-naphthalate | | 0.50 | | 1.00 |
| Diethylamino Hydroxybenzoyl Hexyl Benzoate | | 1.00 | | 1.00 |
| Ethylhexyl salicylate | 1.00 | 0.50 | 2.00 | 1.00 |
| Octocrylene | 2.00 | 1.00 | 3.00 | 2.00 |
| Glycerol | 5.00 | 7.00 | 9.00 | 7.00 |
| Acrylates/C10-30 Alkyl Acrylate Crosspolymer | 0.10 | 0.30 | | 0.10 |
| Sodium polyacrylate | 0.30 | | | |
| Carbomer | | 0.10 | 0.15 | 0.15 |
| Ammonium Acryloyldimethyltaurate/VP | | | 0.25 | |
| Copolymer | | | | |
| Chondrus crispus extract (carrageenan) | | | | 0.10 |
| Phenoxyethanol | 0.50 | 0.50 | 0.40 | 0.40 |
| Piroctone olamine | | | | 0.20 |
| Salicylic acid | 0.05 | 0.10 | 0.01 | 0.20 |
| Benzoic acid | 0.20 | 0.05 | 0.10 | 0.01 |
| Distarch phosphate | | 1.00 | | 0.50 |
| Methyl Methacrylate Crosspolymer | | 0.50 | 0.50 | |
| Glycyrrhiza inflata root extract (licorice root) | | | 0.05 | |
| Hydroxyisohexyl 3-cyclohexenecarboxaldehyde | 0.05 | 0.05 | 0.05 | |
| Citronellol | | 0.05 | | |
| Benzyl salicylate | | | 0.05 | |
| Parfum | 0.20 | 0.20 | 0.20 | |
| Sodium hydroxide | as | as | as | as |
| | required | required | required | required |
| Water | to 100 | to 100 | to 100 | to 100 |

| | Example formulations | | | |
|---|---|---|---|---|
| | 49 | 50 | 51 | 52 |
| Chemical/INCI name | % by weight | % by weight | % by weight | % by weight |
| N-(4-Amino-2-methylquinolin-6-yl)-2-((4- | 0.3 | 0.5 | 0.2 | 1 |
| ethylphenoxy)methyl)benzamide | | | | |
| Potassium cetyl phosphate | 0.20 | 0.20 | 0.25 | 0.20 |
| C12-15 alkyl benzoate | 2.50 | 2.50 | 2.00 | 2.00 |
| Isopropyl palmitate | 2.50 | 2.50 | | 3.00 |
| Isopropyl stearate | | | 2.00 | |
| Caprylic acid/capric acid triglyceride | 2.50 | 2.50 | 1.50 | 2.00 |
| Glyceryl stearate | 1.00 | 1.00 | 1.25 | 1.50 |
| Octyldodecanol | | | 1.50 | |
| Paraffinum Liquidum (mineral oil) | | | | 1.00 |
| Glycerol | 5.00 | 7.00 | 9.00 | 6.00 |
| Bis-Ethylhexyloxyphenol Methoxyphenyl | | 1.0 | | 1.00 |
| Triazines | | | | |
| Titanium dioxide + trimethoxycaprylylsilane | | | 1.00 | 1.00 |
| Phenylbenzimidazole sulfonic acid | 1.00 | 2.00 | 1.00 | 1.00 |
| Butyl Methoxydibenzoylmethane | 1.00 | 1.00 | 2.00 | 2.00 |
| Disodium Phenyl Dibenzimidazole | 2.00 | 2.00 | 1.00 | 2.00 |
| Tetrasulfonate | | | | |
| Ethylhexyl triazone | 1.00 | 1.00 | 2.00 | 1.00 |
| Ethylhexyl methoxycinnamate + BHT | 0.50 | 1.00 | 0.50 | 1.00 |
| Carbomer | | 0.15 | 0.20 | 0.30 |
| Acrylates/C10-30 Alkyl Acrylate Crosspolymer | 0.30 | 0.10 | 0.15 | |
| Xanthan gum | | | 0.15 | 0.10 |
| Methylisothiazolinone | 0.05 | | | |
| Phenoxyethanol | 0.50 | | 0.40 | 0.40 |
| Methylparaben | | | — | — |
| Butylene glycol | | | 3.00 | 3.00 |
| Sodium benzoate | | | | 0.10 |
| DMDM hydantoin | | 0.20 | | |
| Parfum | 0.10 | 0.30 | | 0.30 |
| BHT (tert-butylhydroxytoluene) | 0.05 | | | |
| Tocopheryl acetate | | 0.10 | | |
| Sodium hydroxide | as | as | as | as |
| | required | required | required | required |
| Water | to 100 | to 100 | to 100 | to 100 |

| | Example formulations | |
|---|---|---|
| | 53 | 54 |
| Chemical/INCI name | % by weight | % by weight |
| N-(4-Amino-2-methylquinolin-6-yl)-2- | 0.1 | 0.1 |
| ((4-ethylphenoxy)methyl)benzamide | | |
| Polyglyceryl-3 diisostearate | 1.50 | 1.50 |
| PEG-40 sorbitan perisostearate | 2.50 | 2.50 |
| Lanolin alcohol | 0.50 | 0.50 |
| Paraffinum Liquidum (mineral oil) | 8.00 | 8.00 |
| Cera Microcrystallina | 2.50 | 2.50 |
| Cyclomethicone | 4.00 | 4.00 |
| Isohexadecane | 2.00 | 2.00 |
| Isopropyl palmitate | 5.00 | 5.00 |
| Iodopropynyl butylcarbamate | — | 0.10 |
| Magnesium sulfate | 0.5 | 0.50 |
| Potassium sorbate | 0.10 | 0.05 |
| Benzyl salicylate | 0.10 | 0.01 |
| 1,2-Octanediol | 0.01 | 0.05 |
| Sodium benzoate | 0.10 | 0.01 |
| Glycerol | 7 | 7 |
| Parfum | as required | as required |
| Water | to 100 | to 100 |

| | Example formulations | | | |
|---|---|---|---|---|
| | 55 | 56 | 57 | 58 |
| Chemical/INCI name | % by weight | % by weight | % by weight | % by weight |
| N-(4-Amino-2-methylquinolin-6-yl)-2-((4- | 0.1 | 0.2 | 0.05 | 0.3 |
| ethylphenoxy)methyl)benzamide | | | | |
| Polyethylene glycol (21) stearyl ether | 2.50 | 2.50 | 1.50 | 1.50 |
| Polyethylene glycol (2) stearyl ether | 1.50 | 1.50 | 2.50 | 2.50 |
| Polyethylene glycol (15) stearyl ether | 3.00 | 3.00 | 4.00 | 4.00 |
| Ethylenediaminetetraacetic acid trisodium salt | 1.50 | 1.50 | 1.50 | 1.50 |
| (20% aqueous solution) | | | | |
| Persea gratissima oil (avocado oil) | 0.10 | 0.10 | 0.15 | 0.15 |
| Parfum | as | as | as | as |
| | required | required | required | required |
| 1-(1,2,3,4,5,6,7,8-Octahydro-2,3,8,8- | 0.10 | 0.05 | | 0.05 |
| tetramethyl-2-naphthyl)ethan-1-one | | | | |
| Linalyl acetate | | 0.05 | 0.05 | |
| Citronellol | | | 0.05 | |
| Triethyl citrate | | | | 0.05 |
| Sodium benzoate | 0.10 | 0.02 | | |
| Potassium sorbate | | | 0.10 | 0.01 |
| Silver citrate | 0.10 | | | |
| Water to | to 100 | to 100 | to 100 | to 100 |

| | Example formulations | | | |
|---|---|---|---|---|
| | 59 | 60 | 61 | 62 |
| Chemical/INCI name | % by weight | % by weight | % by weight | % by weight |
| N-(4-Amino-2-methylquinolin-6-yl)-2-((4- | 0.1 | 0.2 | 0.15 | 0.2 |
| ethylphenoxy)methyl)benzamide | | | | |
| Isoceteth-20 | 3.50 | 3.00 | 4.00 | 4.00 |
| Glyceryl isostearate | 2.00 | 2.00 | 2.00 | 2.50 |
| Dicaprylyl ether | | 0.50 | 2.00 | 2.50 |
| Caprylic/capric esters | 2.00 | 1.50 | | |
| Aluminum chlorohydrate | 5.00 | 5.00 | | 3.00 |
| Persea gratissima oil (avocado oil) | | | 0.20 | |
| Polyethylene glycol (150) distearate | 0.50 | 0.50 | 1.00 | 1.00 |
| Glycerol | 4.00 | 2.00 | | 2.00 |
| Butylene glycol | | 3.00 | 1.00 | 2.00 |
| Propylene glycol | 3.00 | | 3.00 | |
| 4-[(Cyclopentylhydroxyphenylacetyl)oxy]-1,1- | 0.05 | 0.10 | | |
| dimethylpiperidinium bromide | | | | |
| Geraniol | | 0.05 | | |
| Ethyllinalool | | | 0.05 | |
| Linalool | | | | 0.10 |
| Parfum | 0.25 | 0.50 | 0.50 | 0.75 |
| Water to | to 100 | to 100 | to 100 | to 100 |

| | Example formulations | | | |
|---|---|---|---|---|
| | 63 | 64 | 65 | 66 |
| Chemical/INCI name | % by weight | % by weight | % by weight | % by weight |
| N-(4-Amino-2-methylquinolin-6-yl)-2-((4- | 0.1 | 0.2 | 0.05 | 0.3 |
| ethylphenoxy)methyl)benzamide | | | | |
| Polyoxyethylene (20) cetyl/stearyl ether | 3.00 | 3.00 | 4.00 | 4.00 |
| Polyoxyethylene (12) cetyl/stearyl ether | 0.50 | 0.50 | | |
| Glyceryl stearate | 3.00 | 3.00 | 3.00 | 3.00 |
| Cetyl stearyl alcohol | 0.50 | 0.50 | | |
| Cetyl palmitate | 0.50 | 0.50 | | |
| Caprylic/capric esters | 4.00 | 4.00 | 3.50 | 3.50 |
| Di-n-octyl ether | 5.00 | 5.00 | 5.00 | 5.00 |
| Polyethylene glycol (150) distearate | | | 1.00 | 1.00 |
| Glycerol | 4.00 | 4.00 | 2.00 | 2.00 |
| 1,2-Hexanediol | 1 | | 0.5 | |
| Sorbic acid | | 0.3 | 0.5 | 0.2 |
| Hexylcinnamaldehyde | 0.05 | 0.10 | | |
| 1-(1,2,3,4,5,6,7,8-Octahydro-2,3,8,8- | | 0.05 | 0.10 | |
| tetramethyl-2-naphthyl)ethan-1-one | | | | |
| 3-Methyl-5-phenyl-1-pentanol | 0.05 | | | 0.05 |
| Parfum | 0.30 | 0.30 | 0.50 | 0.50 |
| Water to | to 100.00 | to 100.00 | to 100.00 | to 100.00 |

| | Example formulations | | | |
|---|---|---|---|---|
| | 67 | 68 | 69 | 70 |
| Chemical/INCI name | % by weight | % by weight | % by weight | % by weight |
| N-(4-Amino-2-methylquinolin-6-yl)-2-((4- | 0.2 | 0.05 | 0.1 | 0.5 |
| ethylphenoxy)methyl)benzamide | | | | |
| Steareth-100 | 1.00 | 1.00 | 1.00 | 1.00 |
| Polyglyceryl-3 diisostearate | 1.60 | 1.60 | 1.60 | 1.60 |
| PEG-45/dodecyl glycol copolymer | 0.80 | 0.80 | 0.80 | 0.80 |
| C20-40 alkyl stearate | 10.00 | 10.00 | 10.00 | 10.00 |
| Capryl ic/capric triglyceride | 3.00 | 3.00 | 3.00 | 3.00 |
| Octyldodecanol | 3.00 | 3.00 | 3.00 | 3.00 |
| Dicaprylyl ether | 4.00 | 4.00 | 4.00 | 4.00 |
| Potassium sorbate | 0.10 | 0.10 | 0.10 | 0.10 |
| Ethylhexylglycerol | 0.10 | 0.10 | 0.10 | 0.10 |
| Butylene glycol | 4.00 | 4.00 | 4.00 | 4.00 |
| Hydroxyisohexyl 3-cyclohexenecarboxaldehyde | 0.05 | 0.05 | 0.05 | 0.05 |
| Parfum | 0.35 | 0.30 | 0.25 | 0.15 |
| Water to | to 100.00 | to 100.00 | to 100.00 | to 100.00 |

| | Example formulations | | |
|---|---|---|---|
| | 82 | 83 | 84 |
| Chemical/INCI name | % by weight | % by weight | % by weight |
| N-(4-Amino-2-methylquinolin-6-yl)-2-((4- | 0.02 | 0.5 | 0.7 |
| ethylphenoxy)methyl) benzamide | | | |
| Glyceryl monostearate | 5.00 | 5.00 | 5.00 |
| Polyethylene glycol (2000) monostearate | 2.00 | 2.00 | 2.00 |
| Stearyl alcohol | 3.00 | 3.00 | 3.00 |
| Cyclomethicone | 4.00 | 4.00 | 4.00 |
| Paraffin oil | 6.00 | 6.00 | 6.00 |
| Trisodium EDTA | 0.20 | 0.20 | 0.20 |
| Aluminum chlorohydrate | 2.50 | 2.50 | 2.50 |
| Sodium benzoate | 0.20 | 0.10 | 0.10 |
| Methylpropanediol | 0.01 | 0.50 | 0.60 |
| 2-Methylpropanediol | 3.00 | 3.00 | 3.00 |
| 2-Ethylhexyl glyceryl ether | 0.50 | 0.50 | 0.50 |
| Benzyl salicylate | — | — | 0.05 |
| Triethyl citrate | — | 0.05 | — |
| Hexylcinnamaldehyde | 0.05 | — | — |
| Parfum | 0.40 | 0.30 | 0.20 |
| Water to | 100 | 100 | 100 |

1.-6. (canceled)

7. An active substance combination, wherein the combination comprises (a) N-(4-amino-2-methylquinolin-6-yl)-2-((4-ethylphenoxy)methyl)benzamide and (b) one or more cosmetically or dermatologically acceptable preservatives and/or preservative auxiliaries.

8. The combination of claim 7, wherein component (b) comprises one or more of 4-hydroxybenzoate, ethylparaben, phenoxyethanol, piroctone olamine, methylisothiazolinone, methylpropanediol, butylene glycol, caprylyl glycol, octane-1,2-diol, ethylhexylglycerol, glyceryl caprylate, propylene glycol, sodium salicylate, sodium benzoate, pentylene glycol, 1,2-hexanediol, DMDM hydantoin, benzyl alcohol, butylene glycol, iodopropynyl butylcarbamate.

9. The combination of claim 7, wherein component (b) comprises one or more of propylene glycol, butylene glycol, 2-methylpropane-1,3-diol, ethylhexylglycerol, 1,2-pentanediol, 1,2-hexanediol, 1,2-octanediol, 1,2-decanediol.

10. The combination of claim 7, wherein component (b) comprises ethylhexylglycerol.

11. The combination of claim 7, wherein component (b) comprises phenoxyethanol.

12. The combination of claim 7, wherein component (b) comprises ethylhexylglycerol and phenoxyethanol.

13. A cosmetic or dermatological preparation, wherein the preparation comprises the active substance combination of claim 7.

14. The preparation of claim 13, wherein the preparation comprises from 0.01% to 20% by weight of component (b), based on a total weight of the preparation.

15. The preparation of claim 13, wherein the preparation comprises from 0.1% to 5% by weight of component (b), based on a total weight of the preparation.

16. The preparation of claim 13, wherein component (b) comprises one or more of 4-hydroxybenzoate, ethylparaben, phenoxyethanol, piroctone olamine, methylisothiazolinone, methylpropanediol, butylene glycol, caprylyl glycol, octane-1,2-diol, ethylhexylglycerol, glyceryl caprylate, propylene glycol, sodium salicylate, sodium benzoate, pentylene glycol, 1,2-hexanediol, DMDM hydantoin, benzyl alcohol, butylene glycol, iodopropynyl butylcarbamate.

17. The preparation of claim 13, wherein component (b) comprises one or more of propylene glycol, butylene glycol, 2-methylpropane-1,3-diol, ethylhexylglycerol, 1,2-pentanediol, 1,2-hexanediol, 1,2-octanediol, 1,2-decanediol.

18. The preparation of claim 13, wherein component (b) comprises at least one of ethylhexylglycerol and phenoxyethanol.

19. The preparation of claim 18, wherein the preparation comprises from 0.1% to 0.6% by weight of ethylhexylglycerol, based on a total weight of the preparation.

20. The preparation of claim 18, wherein the preparation comprises from 0.1% to 1% by weight of phenoxyethanol, based on a total weight of the preparation.

21. The preparation of claim 13, wherein the preparation comprises from 0.001% to 10% by weight of component (a), based on a total weight of the preparation.

22. The preparation of claim 13, wherein the preparation comprises from 0.01% to 1% by weight of component (a), based on a total weight of the preparation.

23. The preparation of claim 13, wherein the preparation further comprises one or more UV filter substances.

24. The preparation of claim 13, wherein the preparation is an emulsion.

25. The preparation of claim 13, wherein the preparation is a hydrodispersion.

26. A method for the treatment and/or prophylaxis of symptoms of intrinsic and/or extrinsic skin aging and for the treatment and/or the harmful effects of ultraviolet radiation on the skin, wherein the method comprises applying to skin in need of treatment and/or prophylaxis the preparation of claim 13.
